(19) United States
(12) Patent Application Publication
(10) Pub. No.: US 2008/0214909 A1
(43) Pub. Date: Sep. 4, 2008

(54) Catheter With Microchannels For Monitoring The Concentration Of An Analyte In A Bodily Fluid

(76) Inventors: Otto Fuerst, Viernheim (DE); Hans List, Hesseneck-Kailbach (DE); Hans-Peter Haar, Wiesloch (DE)

Correspondence Address: BAKER & DANIELS LLP / ROCHE, 300 NORTH MERIDIAN STREET, SUITE 2700, INDIANAPOLIS, IN, 46204, US

(21) Appl. No.: 11/816,770

(22) PCT Filed: Feb. 21, 2006
(86) PCT No.: PCT/EP06/01565

§ 371 Date: Aug. 21, 2007

(30) Foreign Application Priority Data
Feb. 21, 2006 (EP) 2006 001565

(51) Int. Cl. A61B 5/00

(52) U.S. Cl. 600/309; 600/581; 29/428

(57) ABSTRACT

Catheter for monitoring the concentration of an analyte in a body fluid having an implantable distal area (7) for the uptake of body fluids and a proximal area (8) with an outlet opening (14), wherein the catheter comprises a catheter casing (1) having microopenings which are permeable to at least part of the body fluid and at least partially hold back corpuscular components, and a catheter core (2) which is located in the catheter casing (1). The catheter core preferably has a microstructure on its peripheral surface and/or the catheter casing has a microstructure on its inner wall which form a hollow space between the catheter core and catheter casing. Moreover the invention concerns a method for the manufacture of such a catheter.

### RELATED APPLICATION

This application is related to and claims priority to International Patent Application Serial No. PCT/EP2006/001565, filed Feb. 21, 2006, the disclosure of which is expressly incorporated by reference herein.

### BACKGROUND

1. Field of the Invention

The present invention concerns the diagnostic field in which body fluids are taken and analyzed for the presence or concentration of analytes.

2. Description of the Related Art

Numerous methods are known in the prior art for monitoring analyte concentrations in body fluids. On the one hand, there are systems in which blood is removed via a catheter and conveyed to a measuring cell. The document WO 91/16416 which describes a device that can be worn on the arm which removes blood samples by means of a catheter implanted in a blood vessel is mentioned as a representative for this procedure. The sample liquid is conveyed through an essentially closed channel system to an enzyme electrode which is designed for a plurality of measurements. Such a system and also other systems based on continuously measuring, electrochemical sensors have the disadvantage that the sensors have a strong signal drift. This is particularly clear from the document WO 91/16416 with regard to the large effort required for the calibration. In addition devices for ultrafiltration are known in the prior art for which the documents U.S. Pat. No. 4,832,034 and U.S. Pat. No. 4,777,953 are mentioned as examples.

Another procedure for monitoring analyte concentrations is known under the name microdialysis. Representative documents from this field are U.S. Pat. No. 5,174,291, EP 0 401 179 and U.S. Pat. No. 4,265,249. In the arrangements described in these documents flow measuring cells with electrochemical sensors are used. A disadvantage of microdialysis systems is that a perfusion liquid has to be pumped through a hollow catheter. Keeping solutions ready, the pumping process and also the structure of the catheter are technical complications which lead to an increased complexity and to relatively large instruments which make it difficult to carry such devices especially in the case of continuous and mobile monitoring.

The concepts described above for monitoring analyte concentrations in body fluids are based on the assumption that a continuous or at least quasi-continuous measurement at relatively short time intervals is necessary. This explains the exclusive use of continuously operating sensors in flow measuring cells.

Concepts which work discontinuously are also known in the field of analyte concentration monitoring. For example diabetics carry out several discrete measurements during a day to monitor their blood glucose level. For this purpose an incision wound is conventionally firstly made with a lancet and discharged blood is applied to a disposable test element. This blood is analyzed using a suitable device in order to determine the blood glucose concentration. Optically-based systems as well as systems using electrochemical test elements are known in the state of the art. Devices have also been known for some time in which a single disposable test element can be used to generate an incision wound, collect the sample and apply the sample. Such systems for determining blood glucose in interstitial fluid are described for example in the documents U.S. Pat. Nos. 5,746,217, 5,823,973 and 5,820,570. The devices mentioned above have a thin cannula which is inserted into the dermis where it collects interstitial fluid. The cannula conveys the liquid onto a test element. A disadvantage of these systems is that a cannula has to be inserted again for each individual measurement. In addition to the trouble caused by repeated punctures, the user has to carry out a number of handling steps such as inserting a disposable element into an apparatus, triggering the lancing operation, waiting until the result of the analysis is displayed and replacing the test element. Moreover, the user has to carry around the equipment and look for a discreet place for the measurement if he does not want to publicly display his disease.

The prior art WO 02/062210 describes a system with a catheter which combines the advantages of continuously operating systems with those of individual measurements using disposable test elements. A catheter is used which remains implanted between the (at least two) measurements so that it is not necessary to repeat the insertion as is necessary for previous systems using disposable test elements. The problems of previous continuously operating systems which are primarily linked to a use of continuously operating sensors are avoided by using disposable test elements for single use. The document describes a catheter having an implantable region and an outlet opening for removing the interstitial fluid. A first and a second analytical zone are successively contacted with the sample liquid from the catheter and undergo a detectable change when an analyte is present. The analytical zone can be contacted manually with liquid as well as preferably in an automated manner by means of a device. The test zones are for example applied next to one another on a tape and are present in the form of a tape cassette. Dry chemistry test elements can be used for the test elements or test zones which have already proven to be particularly suitable in practice with regard to accuracy and precision as well as having technical advantages for the manufacturing.

U.S. Pat. No. 6,537,243 describes a system having a catheter which has pores which are large enough to allow erythrocytes to pass. The pores are preferably larger than 5 μm in diameter. In addition a needle is at first located in the catheter in order to stiffen it so that it can be inserted into the tissue. The needle seals the inner space of the catheter and must therefore be removed before the measurement.

A disadvantage of the above-mentioned systems is that relatively large amounts of liquid are required because they have fluid channels with correspondingly large inner volumes. As a result of this inner volume a relatively large amount of liquid has to be firstly removed from the body until a first measurement can be carried out even if the sensor itself only requires a small sample volume for a measurement. This is of special importance since the volume flow with which interstitial fluid is removed from the body, has to be very low. If one for example wants to remove liquid over a long period such as more than 24 hours, the amount withdrawn should be approximately of the order of magnitude of 1 nanoliter per minute and square millimeter withdrawal area. This ensures that the physiological processes in the body are negligibly affected and that for example the glucose content of the sample corresponds to the concentration in blood. Even relatively thin catheters having a correspondingly low dead volume accordingly have a very long time delay of frequently more than 30 minutes. A time delay of more than 10 minutes is usually unacceptably long especially for monitoring diabetes.

Furthermore, the selection of a suitable membrane material of the catheter which, on the one hand, should have an adequately high filtering effect and, on the other hand, should be sufficiently permeable and moreover not be blocked after a short period, is critical.

WO 00/22977 describes a minimally invasive sensor system comprising a hollow probe for withdrawing a fluid from tissue for a continuous measurement of substance concentrations in the liquids withdrawn from the tissue by means of a flow sensor. The casing surface of the hollow probe can be perforated and thus be permeable to interstitial fluid. The required sample volume can be reduced if microfluidic elements are used for the flow sensor. In order to stabilize the hollow probe which has a diameter of approximately 0.5 mm, the hollow probe contains a wire or a bundle of fibers as a reinforcement support which can be removed after applying the hollow probe. However, such a reinforcement support has the disadvantage that the liquid volume in the hollow probe is not exactly defined especially when the hollow probe is curved because the wire rests against the inner wall of the hollow probe at some positions and not at others. In addition such an arrangement requires that there must be a relatively large difference between the outer diameter of the reinforcement support and the inner diameter of the hollow probe to ensure that the hollow probe is open for the body fluid. This results in a relatively large dead volume with a corresponding large time delay when determining an analyte concentration. Furthermore, the flow rates of 100 nl/min are relatively large as for example shown by the parallel publication by M. Knoll et al., Sensors and Actuators B 87 (2002) 150-158. Experience shows that no measurements that last longer than 12 or 24 hours can be carried out with such a system.

### SUMMARY OF THE INVENTION

An embodiment of the present invention concerns a catheter for monitoring the concentration of an analyte in a body fluid which is designed for long-term applications in which very little body fluid per time unit is withdrawn. A bodily fluid, such as interstitial fluid, is withdrawn from a depth of greater than 1 mm under the skin surface and a long-term measurement is carried out over at least 12 hours at a flow rate of approximately 10 nl/min with a time lag of less than 10 min.

A catheter according to an embodiment of the present invention has an implantable distal area for the uptake of body fluids and a proximal area with an outlet opening and comprises a catheter casing having micro-openings which are permeable to at least part of the body fluid and at least partially retain corpuscular components, and a catheter core which is located in the catheter casing. The catheter core almost completely fills the inner volume of the catheter casing and in this manner reduces the dead volume of the catheter. However, the catheter core still leaves a hollow space open between the catheter casing and catheter core so that body fluid can be conveyed from the micro-openings in the distal area of the catheter casing to the outlet opening in the proximal area. The catheter core is located in the catheter casing during operation of the catheter.

In the above described system, only a small amount of body fluid has to be withdrawn until a first measurement can be carried out. Furthermore, the reaction time of such a system is very short because only a small volume has to be conveyed until the sample liquid reaches the sensor. Hence the transfer time of the sample liquid from the tissue to the sensor can be very short i.e. the measured concentration value closely corresponds to the current value in the body which, for example, especially in the case of diabetics with strongly fluctuating glucose values is of major importance for their therapy. The micro-openings in the catheter casing which are smaller than blood cells and thus prevent cells from entering the catheter. The size of the micro-openings ensures that the catheter with its very small liquid volume and the resulting very small dimensions of the liquid channels is not blocked. This may be important when the system is used over a long period for example for more than 12 hours and in particular for more than 24 hours.

A catheter according to an embodiment of the invention can be used in systems comprising continuously operating sensors as well as in systems with separate measuring processes. A continuously operating sensor is for example a flow measuring cell. A system with separate measuring processes comprises for example a first and a second analytical zone which are successively contacted with liquid from the catheter and undergo a detectable change when an analyte is present. The analytical zone can be contacted with liquid either manually or automatically by means of a suitable device. Such a system additionally may include an evaluation device to evaluate the analytical zones in order to determine the concentration of the analyte on the basis of the analyte-dependent changes.

The catheter according to an embodiment of the invention is used to monitor analyte concentrations in body fluids. Analytes that can be monitored using the present invention are for example glucose, lactate, electrolytes, active pharmaceutical substances and suchlike. In embodiments of the invention, the bodily fluids sampled are interstitial fluid and blood. When using interstitial fluid, fluid may be obtained from a depth of greater than 1 mm under the skin surface since here there is a good and sufficiently rapid exchange with the system transporting blood.

In embodiments of the invention, catheters are tubes into which body fluid enters and can be withdrawn from an outlet opening as well as bodies with a semi-permeable membrane such that the liquid which enters the catheter only contains a portion of all the body fluid components since the fluid has already been pre-treated i.e. filtered (ultrafiltrate). In embodiments of the invention, catheters having a semi-permeable membrane or a microporous wall separate cells and even larger molecules which could interfere with an analysis or block the catheter. Therefore, membranes or microporous walls which have a pore size below 500 nm, for example 100 nm, may be used.

The cross-section of the catheter can be round or elliptical, it can, however, also be angular e.g. rectangular, hexagonal or octagonal and the cross-sectional geometry can change over the length of the catheter.

Within the scope of this invention the term catheter is not only used for the part that is implanted into the body, but rather the term catheter should also encompass the fluid connections belonging to such a part and other connected parts. In the simplest case the catheter can consist of a thin hollow needle or a tube, one end of which is inserted into the body and from the other end of which, the outlet opening, the body fluid flows out. A tube or suchlike can be coupled to such a catheter in order to move the outlet opening to the corresponding end of the tube. The structure and operating mode of suitable catheters is described in more detail in connection with the figures. A so-called insertion device may be utilized to insert the implantable area of the catheter into the body. This also allows the implantable area to be designed with a very small diameter down to for example 100 μm. Even materials such as steels are flexible in these thickness ranges. Suitable insertion devices for flexible and also for rigid arrangements are known in the prior art. At this point reference is made to U.S. Pat. No. 3,651,807; EP A 0 366 336; WO 95/20991 and WO 97/14468 as examples where suitable insertion devices are described.

A catheter according to the invention can be used in a system comprising two or more analytical zones which undergo a detectable change after contact with liquid removed from the outlet opening. Diverse forms of suitable analytical zones are known from the field of disposable test elements. For example, a design of an analytical detection zone is described in U.S. Pat. No. 6,029,919. With regard to the layers of the test element it is of course also possible to use less complex test elements. Electrochemical test elements can also be used within the scope of the invention. Electrochemical test elements are described for example in U.S. Pat. No. 5,288,636.

The use of the terms “analytical zone” or “detection area” in contrast to “test element” makes it clear that the analytical zones do not necessarily have to be elements that are separate from one another but rather that the test zones can indeed be arranged on the same body or test element. In an embodiment of the system according to the invention a tape is used in which a test chemistry is arranged in tape form and areas of the tape are arranged next to one another can be contacted with liquid emerging from the catheter. This also makes it clear that the term analytical zone is not limited to embodiments in which the analytical zones are predetermined but rather that embodiments in which the respective analytical zone may be determined by contact with liquid. This allows positioning problems to be largely circumvented. However, on the other hand, it is also possible, to use test elements that are separated from one another, each of which provides one or more analytical zones.

The analytical zones can be contacted with liquid for example by bringing together the analytical zones and the outlet opening of the catheter. Bringing together in this sense primarily means that analytical zones are moved to the outlet opening so that they can pick up liquid there. However, for example in the case of outlet openings which are located on a flexible tube, it is also possible to move the outlet opening to an analytical zone in order to make contact. The term “bring together” should also encompass processes in which analytical zones for example in the form of a tape are moved past the outlet opening (in contact with the outlet opening or in the vicinity thereof) in order to apply liquid to the analytical zones.

Embodiments are possible in which liquid is removed from the catheter by contact alone. This can be achieved above all with absorbent or capillary-active analytical zones. The system may be configured such that liquid from the outlet opening only emerges when negative pressure is applied. In this manner simply by controlling the pressure conditions in the system, it is possible to control the application of liquid to the analytical zone.

In an embodiment of the invention, the outlet opening of the catheter is not in direct contact with the sensory element. The liquid collects at the outlet opening and is transferred at a defined time onto an analytical zone e.g. by a relative movement of the analytical zone and outlet opening or by means of the fact that the liquid drop touches the analytical zone when it reaches a certain size and the capillary action of the analytical zone has the effect that the drop is transferred onto the detection area. Subsequently the contact with the sensor is broken off again.

The distal area of the catheter describes the area which can be implanted into the body for example by inserting the catheter into the dermis of the abdominal wall. In order to facilitate the insertion, the distal end of the catheter can have a pointed tip. The tip can for example have a circular pointed taper such as is the case with a pin or acupuncture needle or it can have ground surfaces as is for example the case with a lancet. Body fluid is collected from the surrounding body tissue in the distal area and transported inside the catheter into the proximal area of the catheter. The proximal area has an outlet opening from which the body fluid discharges from the catheter and for example contacts an analytical zone.

A system for monitoring the concentration of an analyte can additionally comprise an evaluation unit for evaluating the analytical zones after contact with liquid. Such evaluation devices are well-known in the prior art, for example for blood sugar measuring instruments. Photo optical or electrochemical measuring procedures are used for this. Photo optical methods are for example reflection photometry, absorption measurement or fluorescence measurement, and electro-chemical methods are for example potentiometry, amperometry, voltammetry or coulometry. Reference is made to the document U.S. Pat. No. 4,852,025, as an example in which a conversion of reflection-photometric measurements into concentration values is described. Such an evaluation device comprises a light source for illuminating an analytical zone, a detector for detecting radiation reflected from the analytical zone and an electronic circuit to convert the detector signals into analyte concentrations.

An embodiment of the invention concerns a catheter wherein the core of the catheter has a microstructure on its peripheral surface and/or the catheter casing has a microstructure on its inner wall which form a hollow space between the catheter core and catheter casing. This structure which generates a hollow space can for example be formed by elevations or depressions in the catheter core and/or catheter casing. Thus, the depressions or elevations may be present only in the catheter core or elevations or depressions may be present in the casing in addition to the depressions in the core. Furthermore, of course other combinations of elevations and depressions are also possible. The microstructures in the catheter core and catheter casing can, on the one hand, lie on top of one another but they can also be offset relative to one another. They can be offset either over the circumference and/or over the length of the catheter. The catheter core or catheter casing may have different microstructures such as elevations and depressions in the different areas of the catheter and in particular in the proximal area and in the distal area. For example the catheter core has a first microstructure in its distal area and a second microstructure in its proximal area which has a different shape to that of the first microstructure. In particular the distal area of the catheter can have a plurality of microstructures e.g. distributed over the entire circumference whereas for example only a few microstructures are present in the proximal area. The microstructures according to the invention have the effect that, on the one hand, the catheter core can make a tight seal at its contact area with the catheter casing and a liquid volume is only present in the channels that are formed by the microstructures. In this manner the liquid inner volume i.e. the dead volume of the catheter is considerably reduced and the volume is exactly defined so that the transfer volume and transfer time are known and can be taken into account for the measurement. This for example improves the interpretation of the measured values since, if the reaction time of the measurement system is known, for example the reaction to an insulin injection can be better observed and thus the therapy can be optimized.

In a further embodiment the microstructures consist of micro-channels or of regular microbodies in the form of truncated cones, truncated pyramids or spherical calottes. For example a microstructure generating a hollow space extends over the entire peripheral surface in the distal area whereas the hollow space generated in the microstructure is present in a part of the peripheral surface in the proximal area of the catheter. The cross-section of the catheter casing can be round or elliptical and the catheter core lies essentially coaxially inside the catheter casing. Moreover, the catheter casing can also have an essentially angular rectangular profile and the catheter core lies within and has a similar profile. Furthermore, it is also possible to have a combination of different profiles for example a round catheter casing cross-section with an angular catheter core.

In addition fine fibers e.g. carbon fibers, glass fibers, fine stainless steel or titanium wires may be located between the catheter core and the catheter casing which thus can generate a hollow space. In this case the core and/or the casing can be provided with or without elevations or depressions. For example the core can merely have one or more longitudinal grooves, the casing is for example unstructured and a fiber is wound around the core in the distal area. In this case the longitudinal grooves in the distal area serve as a collecting channel and as a transfer channel in the proximal area. In this embodiment the structuring of the core is constant over its entire length which simplifies a continuous production process. Moreover, the longitudinal grooves can be more easily integrated into such a process as structures which run radially.

The micro-channels can comprise longitudinal grooves which run axially and/or transverse grooves which run radially. Moreover, other shape forms are also possible for example micro-channels that are formed in a screw shape. The micro-channels of the catheter core and/or catheter casing can for example have approximately 8 to 100 axial grooves and approximately 1 to 10 transverse grooves especially in the distal area of the catheter.

In an embodiment of the invention, the catheter core is built monolithically i.e. in one piece from a single component. For example a metal or plastic wire can serve as the catheter core. The micro-channels in the catheter core and/or catheter casing are manufactured using microtechnology where the width of the micro-channels is for example approximately 5 to 20 μm and the depth of the micro-channels is also approximately 5 to 20 μm. In another embodiment of the invention the catheter core fills more than 95% of the hollow space of the catheter casing so that especially in the proximal transfer area of the catheter the dead volume of the sample liquid is very small.

The micro-openings in the catheter casing refer to pores through which body fluid from the surrounding body tissue can enter the catheter. In this case the pore size is selected such that the openings are permeable at least for a part of the body fluid and at the same time corpuscular components are at least partially held back. The retention of cells and deposits from the tissue should prevent micro-channels, which have the approximate size of cells, from being blocked by them. The permeability to body fluid and in particular interstitial fluid is necessary so that the body fluid can be collected from the body and transported to a test element. The micro-openings for example have a diameter of approximately 0.1 μm to 1 μm and may have a diameter approximately 0.1 μm. The size distribution of the pores can be very constant, but the pore size can also be distributed over a wide range. The distribution of the micro-openings over the catheter casing can be in regular patterns or irregular.

The invention also includes a system for monitoring the concentration of an analyte in a body fluid which comprises a catheter according to the invention with an implantable distal area for the uptake of body fluids and a proximal area with an outlet opening wherein the catheter casing has micro-openings. In addition the system has a detection area for detecting an analyte in the body fluid. The detection area undergoes a detectable change for example after contact with body fluid if an analyte is present in the fluid. An embodiment of such a system comprises a first and a second analytical zone where the analytical zones are for example areas of a continuous test element such as a tape, for example. Another embodiment of such a system additionally comprises means for generating a negative pressure in the catheter. Application of a negative pressure in the catheter can support the transport of the body fluid in the catheter or allows it to be exactly controlled and thus allows the transport rate to be exactly adjusted.

Furthermore, the invention concerns a method for producing a catheter for monitoring the concentration of an analyte in a body fluid which comprises the steps of generating micro-openings in a catheter casing which are permeable at least for a part of the body fluid and retain corpuscular components at least partially and assembling the catheter casing around a catheter core. Additionally the method for producing the catheter can comprise the step of introducing micro-channels into a catheter core and/or the catheter casing using means of microstructure technology.

According to the invention, simple production processes suitable for series production can be used. For example the catheter can be constructed by microassembly injection molding. In this process the catheter core is placed in the mold as an insert which may be made of steel or optionally of titanium or suitable alloys. A catheter casing made of plastic is extrusion coated around the inserted catheter core. The plastic that is used must be suitable for microassembly injection molding and have a high molding precision. The catheter tip can either be formed in a subsequent process step or when the catheter is cut into pieces. For example the front of an injection molded is ground to a catheter tip section on the insert piece that is not yet separated. Subsequently the catheter needle is separated in the following section and processed further. The catheter casing can for example be pre-manufactured as a tube-shaped membrane or if it is a flat membrane, it can for example be glued or welded to form membrane tubes. The membrane tubes can be pulled over the catheter core or in the case of flat membranes they are wound around the catheter core and are glued or welded on the core to form a tube. The catheter casing can for example be fixed or additionally glued onto the catheter core by means of holding grooves or be fixed by thermal shrinking.

A method for introducing micro-channels into the catheter core is microform ablation with a laser which is for example described in the “Nachrichten Forschungszentrum” Karlsruhe volume 34, 2-3 2002, pages 210-220, the entire disclosure of which is hereby incorporated by reference herein. ND/YAG lasers or excimer lasers can for example be used for the laser structuring which enable a structure resolution of about 1 μm. Further microstructures can subsequently optionally be applied to the catheter core by electrochemical etching.

Another method is to produce the micro-channels in the catheter core by photolithographic material build-up. In this process a radiation-sensitive polymeric lacquer a so-called resist is applied to the catheter core and the chemical solubility of the resist is changed by selective irradiation for example with UV light. As a result the resist has a radiation-dependent considerably lower or considerably higher resistance to solvents. This lithographic structuring allows a selective removal of structures that have been exposed differently and a subsequent further processing of the exposed surface of the support material. The exposure is carried out by means of special mask-based methods. In order to build up material the catheter core receives a metal deposition over the whole area after the development together with the remaining photoresist. After the remaining resist together with its overlying metal layer has been removed, the metal layer remains structured on the catheter core.

Furthermore, photolithographic methods can also be used to produce microstructures on the catheter core by means of wet chemical or dry chemical etching by material ablation.

Another subject matter of the invention describes a method for monitoring the concentration of an analyte in a body fluid which comprises the steps of taking up body fluid in an implantable distal area of a catheter which comprises a catheter casing with micro-openings which are permeable to at least a part of the body fluid and which at least partially hold back corpuscular components, transporting the body fluid from the distal into a proximal area of the catheter, contacting the body fluid with the detection area and detecting the analyte in the detection area.

The distal area of the catheter has a catheter casing with micro-openings which are permeable to at least a part of the body fluid and at least partially hold back corpuscular components. In addition negative pressure can be applied to transport the body fluid from the distal to the proximal area of the catheter. For example with such a method body fluid can be transported in the catheter at a feed rate of approximately 10 nl/min and the body fluid can be conveyed in less than about 15 minutes, and in embodiments between approximately 5 to 10 minutes from the distal to the proximal area of the catheter.

### BRIEF DESCRIPTION OF THE DRAWINGS

The above-mentioned and other features of this invention and the manner of obtaining them will become more apparent and the invention itself will be better understood by reference to the following description of embodiments of the present invention taken in conjunction with the accompanying drawings, wherein:

FIG. 1A depicts a cross sectional view of a catheter according to an embodiment of the present invention;

FIG. 1B depicts a portion of the catheter depicted in FIG. 1A magnified for illustrative purposes;

FIG. 1C depicts a perspective view of the catheter depicted in FIG. 1A;

FIG. 2A depicts a perspective view of an catheter core according to an embodiment of the present invention;

FIG. 2B depicts a perspective view of the catheter core shown in FIG. 2A, with a portion of the casing omitted for illustrative purposes;

FIG. 3 depicts a cross section view of the catheter depicted in FIG. 1 taken near the proximal end of the catheter;

FIG. 4 depicts a perspective view of a catheter according to an embodiment the invention;

FIG. 5 depicts a perspective view of a catheter according to an embodiment of the invention;

FIG. 6 depicts a portion of FIG. 5 magnified for illustrative purposes;

FIG. 7 depicts a system according to an embodiment of the invention for monitoring the concentration of an analyte in a body fluid;

FIG. 8 depicts a top view of a test element tape;

FIG. 9 depicts a mode of operation of a catheter representing an embodiment of the present invention;

FIG. 10 depicts an exploded perspective view of a system configured for use with embodiments of the catheters according to the present invention;

FIG. 11 depicts a perspective view of another embodiment of a system configured for use with embodiments of catheters according to the present invention; and

FIG. 12 depicts another embodiment of a monitoring system configured for use with a catheter of the present invention.

Corresponding reference characters indicate corresponding parts throughout the several views. Although the drawings represent embodiments of various features and components according to the present invention, the drawings are not necessarily to scale and certain features may be exaggerated in order to better illustrate and explain the present invention. The exemplifications set out herein illustrate embodiments of the invention, and such exemplifications are not to be construed as limiting the scope of the invention in any manner.

### DETAILED DESCRIPTION OF EMBODIMENTS OF THE INVENTION

For the purposes of promoting an understanding of the principles of the invention, reference will now be made to the embodiments illustrated in the drawings, which are described below. It will nevertheless be understood that no limitation of the scope of the invention is thereby intended. The invention includes any alterations and further modifications in the illustrated devices and described methods and further applications of the principles of the invention, which would normally occur to one skilled in the art to which the invention relates.

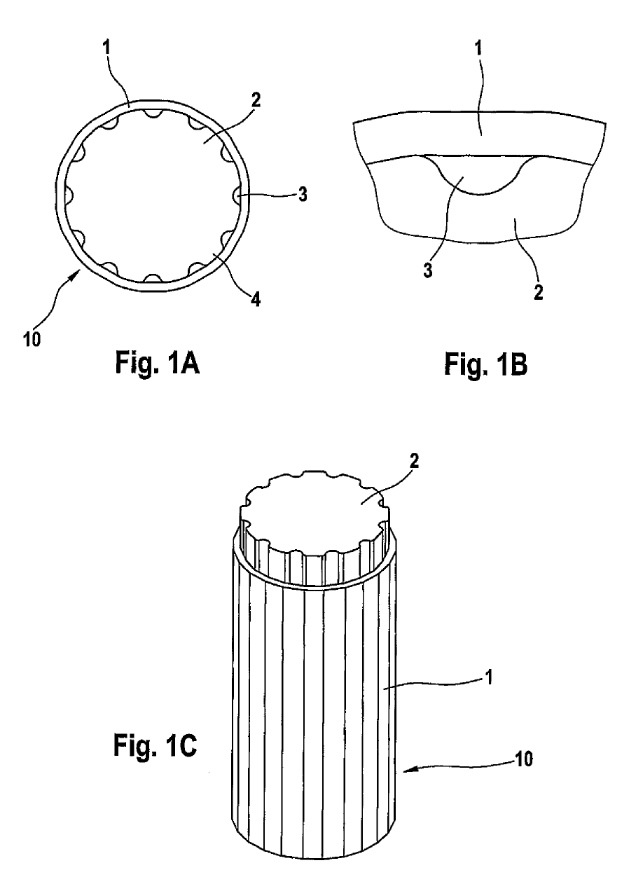

FIG. 1A shows a cross-section through an embodiment of a catheter (10) according to the present invention. The catheter (10) is composed of an assembly of a catheter casing (1) which in this case comprises a thin tube membrane and a catheter core (2) which for example comprises a metal wire into the circumference of which depressions (3) are incorporated. These depressions can either be produced by material ablation or by generating elevations (4) for example by applying material to the catheter core (2). The axial grooves (3) form micro-channels in the assembly of catheter casing (1) and catheter core (2) in which the body fluid that passes through the micro-openings through the catheter casing (1) from the surrounding tissue, is collected. FIG. 1B shows an enlarged detail of FIG. 1A which shows how the micro-channels (3) having a defined inner volume are formed by the catheter casing (1) resting tightly on the catheter core (2). FIG. 1C shows a perspective view of an embodiment of a catheter according to the present invention.

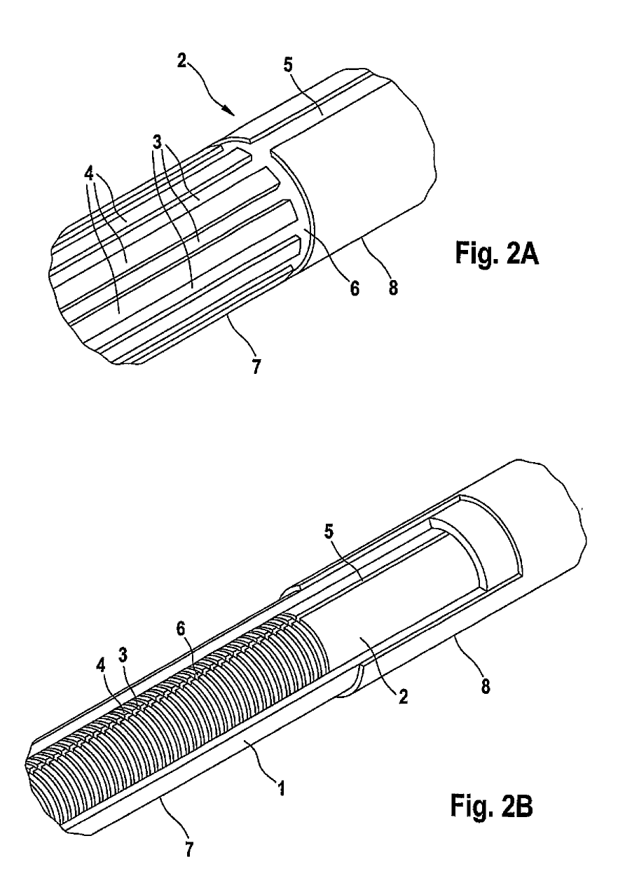

FIGS. 2A and 2B show embodiments of a catheter core (2) according to the present invention at the junction between the distal area (7) and the proximal area (8). The catheter casing (1) which in the assembly is located over the catheter core (2), has a plurality of micro-openings in the distal area (7) through which the body fluid and in particular interstitial fluid from the surrounding body tissue enters the micro-channels (3) which are for example formed by surrounding elevations (4). The fluid from the individual channels (3) is brought together, collected and transported in a transport channel (5) to the outlet opening of the catheter (10) in a collecting channel which is shown in FIG. 2A by a transverse groove (6) as an example and in FIG. 2B by a longitudinal groove (6) as an example. The transport channel (5) is located in the proximal area (8) of the catheter (10). In this area the catheter casing (1) has no micro-openings since in this area the body fluid is not collected from the surroundings but rather should be transported from the distal area (7) to the outlet opening. In the distal area (7) of the catheter (10) the micro-channels or microstructures are arranged such that an inner liquid volume can be provided which is in diffusive exchange with the outer surroundings so that it for example reflects the glucose concentration of the surrounding interstitial fluid. The catheter core (2) and/or catheter casing (1) in the distal area (7) consist either only of axially running longitudinal grooves (3) (see FIG. 2A) or of transverse and longitudinal channels (3) (see FIG. 2B), micro-channels or other structures resulting in a hollow space. The function of the proximal area (8) of the catheter (10) is to transport the body fluid collected in the distal area (7) to the outlet opening (14) using the smallest possible transfer volume. An elevated transfer volume or dead volume, on the one hand, increases the volume requirement of the system and, on the other hand, increases the reaction time of the system. The extremely small transfer volume of the catheter according to the invention in the transport channel (5) allows a real-time measurement of the glucose concentration for example with a transfer time of less than 10 min.

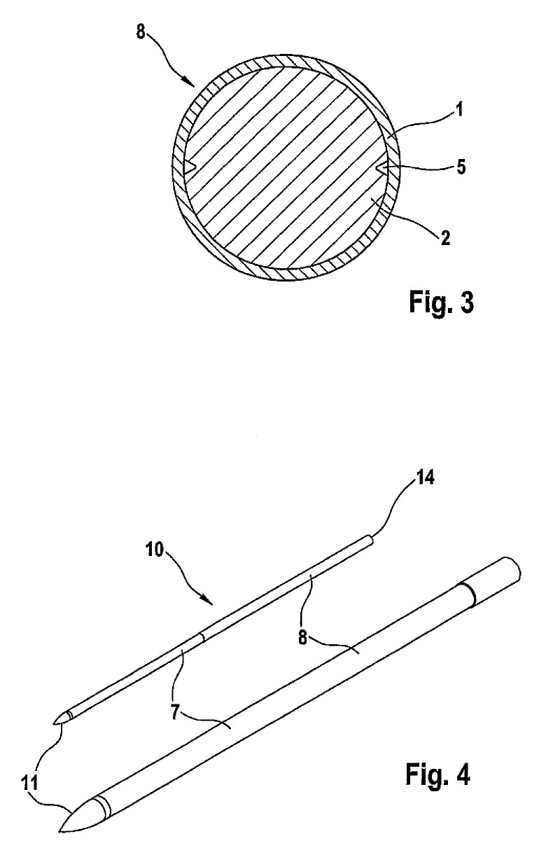

FIG. 3 shows a cross-section through a proximal area of a catheter (8) according to the invention. The contact areas of the catheter casing (1) make a tight seal against the catheter core (2) so that body fluid is only transported through the transport micro-channels (5) which is why the transfer volume can be considerably reduced.

FIG. 4 shows a perspective view of a catheter (10) according to the invention. The tip (11) of the catheter (10) is introduced into the body tissue. In the distal area (7) there are collecting micro-channels (not visible) which collect the body fluid that enters through the micro-openings in the catheter casing (1). In the proximal area (8) the collected body fluid is transported in microtransport channels which are formed by depressions or elevations in the catheter core or catheter casing. The proximal area (8) of the catheter casing (1) has no micro-openings. In this area the body fluid is transported to the outlet opening of the catheter outlet (14). A catheter (10) according to the invention can for example have the following dimensions: a catheter length of approximately 5 to approximately 25 mm where the distal area (7) for example has a length of approximately 10 to approximately 15 mm; a catheter diameter of approximately 0.2 mm to approximately 1 mm; a total inner volume of the catheter of less than approximately 100 nl where the transfer volume from the distal area (7) which is in exchange with the tissue up to the outlet opening of the catheter (10) is less than approximately 50 nl; and a catheter casing with micro-openings in the distal exchange area (7) where the catheter casing comprises a plastic membrane such as polycarbonate, for example, with pores of less than approximately 0.5 μm, and in some embodiments less than approximately 0.1 μm and the porosity of the membrane may be more than approximately 5%. The catheter core comprises a plastic or metal wire. A structuring that yields a hollow space is applied by microtechnological methods e.g. photolithographic methods, which in the distal area (7) together with the micro-openings in the catheter casing are used for exchange with the tissue for example by means of a plurality of transverse and/or longitudinal micro-channels, and in the proximal transfer area (8) they are used to transport the sample liquid for example by means of a longitudinal micro-channels, numbering generally from 1 to 7. The distal area (7) of the catheter casing has micro-openings where these pores may be so small that at least part of the body fluid is retained and in particular blood cells are held back to prevent a blockage of the catheter (10). On the other hand, the pores must be so large that body fluid and in particular interstitial fluid can diffuse unhindered through the membrane and thus be collected. The proximal area (8) of the catheter casing has no micro-openings and only serves to form transfer micro-channels in the assembly with the catheter core through which body fluid collected in the distal area (7) can be transported to the outlet opening of the catheter (10). The distal end of the catheter (10) has a tip (11) which is for example formed by a pointed catheter core and thus facilitates the insertion of the catheter (10) into the body tissue.

Using a catheter according to the invention having a diameter of approximately 0.4 mm and a length of approximately 10 to approximately 15 mm it is for example possible to continuously convey interstitial fluid over 24 hours at a delivery rate of approximately 10 nl per minute where an inner volume of less than approximately 100 nl results in a delay period of less than approximately 10 min. The surface area which exchanges with the tissue is about 10 mm² in size so that at a delivery rate of approximately 10 nl per minute or about 1 nl per minute per mm² interstitial fluid is removed from the tissue. This volume flow is small enough to not affect the liquid balance in the tissue, and, on the other hand, large enough to detect the glucose concentrations in the sample volume. Using this configuration it is for example possible to apply approximately a 50 nl sample volume to an analytical zone and measure it there about every 5 minutes. In the case of a catheter that is thinner or thicker or shorter or longer, the values are scaled accordingly. The microstructure in the distal area which generates the hollow space can for example amount to about 30% to almost 100% of the intermediate surface to enable the best possible diffusion of body fluid from the tissue into the catheter. In contrast the microstructure in the proximal area has a minimal inner volume in order to keep the required transfer volume as low as possible.

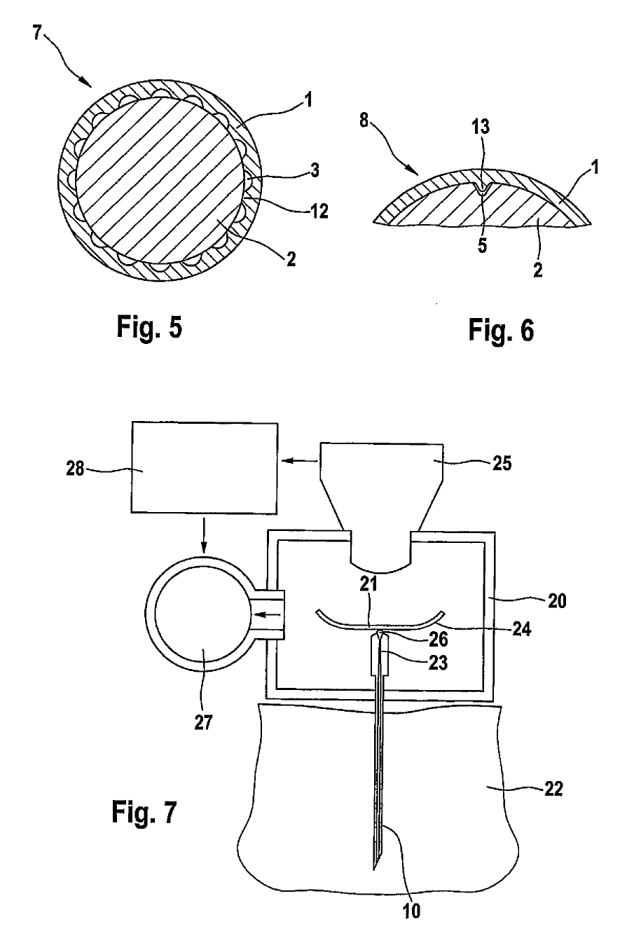

FIG. 5 shows a cross-section through the distal area of a catheter (10) according to the invention where the collecting micro-channels (3) are formed by depressions (12) in the catheter casing (1). The catheter core (2) in this case has for example no microstructures.

FIG. 6 shows a catheter according to the invention in which the catheter casing (1) as well as the catheter core (2) have microstructures. A section of the cross-section of the proximal area of a catheter is shown in the figure. The catheter casing (1) encloses the catheter core (2) making a substantially tight circumferential seal up to the area of the microtransfer channels (5) which are formed by depressions in the catheter core (2). Additional elevations (13) in the catheter casing (1) protrude into the transfer channel (5) and thus further reduce the transfer volume. In addition depressions in the catheter casing (1) and elevations in the catheter core (2) are also conceivable in order to form the micro-channels.

FIG. 7 shows a system according to the invention for monitoring the concentration of an analyte in a body fluid. In this case negative pressure within a housing (20) is utilized in order to collect sample liquid from a catheter (10) and deposit it on an analytical zone (21). The catheter (10) transports the liquid from the subcutaneous tissue (22) to the analytical zone (21). The catheter (10) can comprise a reducer (23) in order to make the liquid flow independent of the resistance to fluid flow of the tissue. A segment of a test element tape (24) which contains the analytical zones (21) is shown schematically. The tape (24) is for example part of a roll which is unrolled in such a manner that an unused analytical zone is moved to the contact site of the catheter (10) for each measurement. The analytical zone enables a photo optical detection of the analyte and moreover it absorbs the sample. The used test elements including the liquid samples thereon are disposed of after one or two days as part of a disposable module e.g. a magazine. An optical reading module (25) which comprises a light source and a photo sensor is positioned in such a manner that it detects the color change in the analytical zone when this zone is wetted by the sample. The sample (26) is applied through an outlet opening of the catheter (10) onto the front side of the analytical zone whereas the optical evaluation for determining the analyte concentration in the sample liquid is carried out from the rear side of the test element. A vacuum pump (27) reduces the pressure within the housing (20) and thus draws liquid out of the catheter (10). A control unit (28) regulates the pumping time and pressure so that the sample liquid flows out of the catheter (10) and wets the analytical zone in a defined manner.

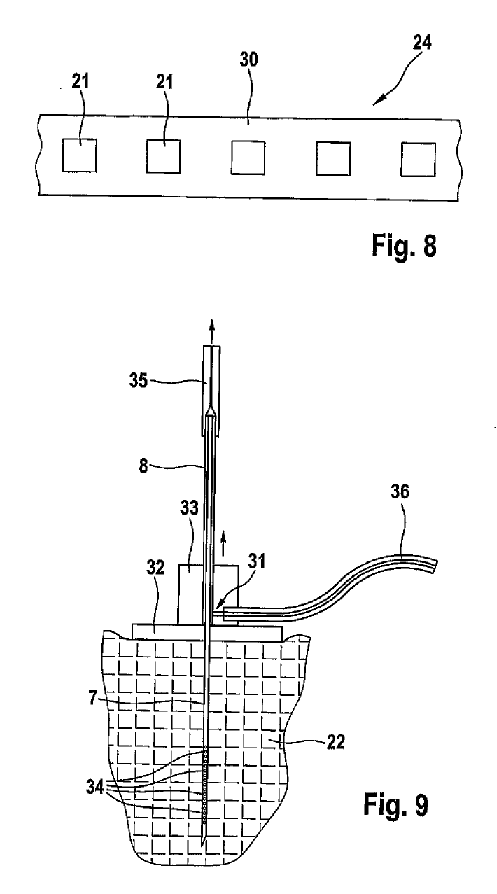

FIG. 8 shows a test element in the form of a test element tape (24) which for example can be used in a system for the continuous measurement of the glucose concentration in interstitial fluid comprising a catheter according to the invention. The tape has a support (30), for example made of polyethylene, onto which the reagent for the analytical zones (21) is applied. The individual analytical zones (21) are applied separately to the support so that in each case one analytical zone (21) can be used for a test. The tape (24) is moved to the outlet opening of the catheter in such a manner that the sample liquid wets an analytical zone (21). Subsequently the concentration of the analyte is determined and after the measurement is completed, the tape (24) is transported further to such an extent that a new, unused analytical zone (21) is moved to the catheter.

FIG. 9 shows an example of the construction and mode of operation of an embodiment of a catheter according to the present invention. The catheter comprises a distal area (7) which is implanted in the tissue (22) of a patient. The catheter is manufactured from stainless steel or any other suitable material and has an outer diameter of approximately 500 μm, an inner diameter of approximately 100 μm and a length of approximately 7 mm. Plastics can also for example be used as an alternative to stainless steel. A proximal area (8) adjoins the distal area (7) of the catheter. An outlet tube (36) is located at an outlet opening (31) of the catheter above the junction area between the implanted area (7) and the proximal area (8). The catheter arrangement is attached to the body surface by a disk-shaped holder (32). Adhesive can be provided on the underside of the holder (32). In order to further stabilize the arrangement, a connecting element (33) which in particular ensures a substantially fluid-tight coupling of the outlet tube (36) is located above the holder (32). The body fluid enters the catheter through the implanted area (7) and is conveyed by capillary forces and by negative pressure into the proximal portion (8) of the catheter. The implanted portion (7) has one or more openings (34) to enable body fluid to enter. The length of the implanted portion (7) and the position of the inlet openings (34) can be used to determine from which depths body fluid is conveyed. It has proven to be advantageous to convey body fluids from depths of more than 1 mm, and in embodiments to utilize a depth range of approximately 3 to approximately 10 mm. It was found that the uppermost skin layers (epidermis and dermis) which together have a thickness in the region of about 1 mm only have a weak mass transfer with the inside of the body and in particular with the bloodstream.

The body fluid rises in the proximal portion (8) of the catheter. For this purpose it is advantageous to hydrophilize the inner region of the hollow needle which is intended to be wetted by the sample liquid. This can be achieved in metallic hollow needles for example by applying a hydrophilizing coating. If the capillary forces are not sufficient, then a negative pressure can be applied to convey or exactly regulate the delivery rate of body fluid from the inside of the body.

An air vent attachment (35) which enables the air displaced by the body fluid to flow out, is provided at the upper end of the hollow needle. The air vent attachment (35) may be made hydrophobic to prevent body fluid from escaping over the catheter. The air vent attachment (35) can for example be a plastic tube made of a hydrophobic polymer e.g. polyethylene. Another important function of the air vent attachment (35) is to limit evaporation from the hollow needle to prevent blockage of the arrangement by dried up liquid.

At first only the inner space of the hollow needle fills but not the connecting tube (36). This can for example be achieved by a connecting tube that has a hydrophobic inner wall. The proximal portion (8) is emptied by applying a negative pressure to the connecting tube (36). After this space has been emptied, air is sucked in so that the body fluid moves in the form of a bolus through the connecting tube onto an analytical zone (not shown) which is contacted with the outlet tube (36). After the upper catheter inner space has been emptied, it can slowly be filled up again by liquid which flows in afterwards from the implanted portion (7). In order to monitor the glucose concentration in humans it is sufficient to carry out measurements at an interval of about 5 minutes so that the time period required to fill up the proximal catheter area is relatively uncritical.

The system shown in FIG. 9 operates in a batch wise manner and the volume provided by an evacuation can be adjusted by the volume in the upper needle area (8). As an alternative to this procedure it is also possible to withdraw liquid onto a test zone directly from an implanted needle by for example contacting the test zone with the outlet opening.

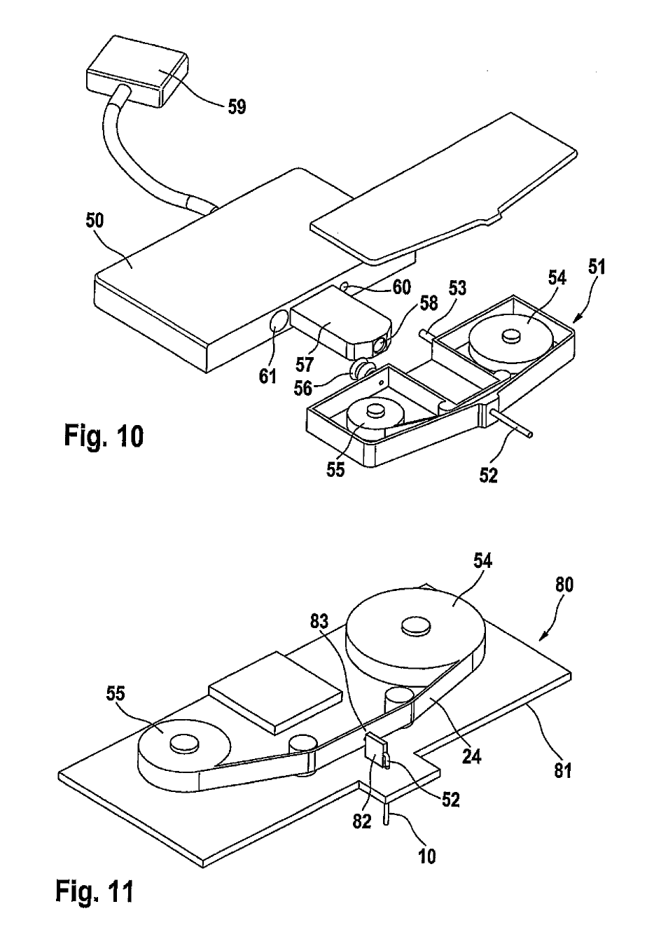

FIG. 10 shows a system for monitoring concentrations in which the catheter according to the invention can be used. The system includes a measuring unit (50) and a disposable unit (51), in which analytical zones are arranged on a test element tape. The connecting tube (52) which can be connected to the catheter (10) is shown on the front side of the disposable unit (51). The unit (51) has a closed design so that a negative pressure compared to the outer space can be applied to its inner space by means of a vacuum connection (53). Two reels are located in the inner space of the unit (51), the first of which, the dispenser reel (54) has a tape-shaped test element wound onto it. The tape is wound from the first reel (54) passing behind the outlet of the tube (52) and is wound onto the second reel, the waste reel (55). Use of an absorbent analytical tape is considered within the scope of the invention since liquid can be taken up and absorbed thus avoiding contamination of the inner space and ensuring a hygienic disposal of the fluids. In order to operate the reel mechanism, the unit (51) has a rubber bush (56) in which a plunger moves, which is operated by the measuring unit (50) and causes the analytical tape to be wound stepwise onto the reel (55). The measuring unit (50) is equipped with an optical head (57) which is inserted into a recess of the disposable unit (51). The optical head (57) has a light source for illuminating the analytical tape and a detector to record reflected radiation. For this purpose an optical window (58) is provided on the front side of the optical head (57). Since the analytical tape runs in an area that is sealed from the outer space and to which negative pressure can be applied, a transparent window is provided in the unit (51) between the analytical tape and optical head. The measuring unit (50) additionally has an electronic evaluation unit to determine analyte concentrations on the basis of the reflected radiation. The determined results can for example be shown directly on a display or they are passed onto a data processing unit (59) in order to be displayed by or transmitted from the processing unit. The measuring unit (50) additionally has a connector (60) for the tube (53) as well as a pump connected to the connector which can be used to pump air out of the disposable unit (51). Furthermore, the measuring unit (50) has a connector (61) for the rubber flange (56) and a drive mechanism for a plunger which moves in the flange. After the measuring unit (50) and disposable unit (51) have been connected together and with a catheter, analyte concentrations are monitored as described below.

Negative pressure is applied to the disposable unit (51) by the pump of the measuring unit (50) such that body fluid that has collected in the catheter is sucked into the unit (51) through the tube (52) and reaches the tape-shaped test element. A reflection-photometric evaluation of the analytical zone is carried out with the evaluation optics (57) and the measured result is converted into a concentration value of the analyte concentration. In addition it is also possible to monitor the fluid application onto the analytical zone and if a sufficient amount of fluid is detected, contact of the analytical zone with liquid can be interrupted for example by removing the negative pressure. After the measurement has been carried out, usually several minutes elapse until some tape is wound onto the waste reel (55) by actuating the drive mechanism and thus a fresh test zone is moved into the vicinity of the outlet opening of the tube (52). Liquid can now be conveyed by again applying a negative pressure.

A similar disposable unit (80) to the disposable unit (51) shown in FIG. 10 is shown in FIG. 11. In this disposable unit (80), the catheter (10) that can be implanted in the body is already integrated. The implantable distal area is arranged perpendicular to the base (81) of the disposable unit (80). This enables the hollow needle (10) to be implanted directly in the body by pressing the base (81) of the disposable unit (80) onto a body surface thus simplifying the handling. The catheter (10) joins the connecting tube (52) which is held by a holder (82). The tape-shaped test element (24) is guided past the outlet position of the connecting tube (52) thus resulting in a sample application point (83). If for example one measurement per 5 minutes is carried out, approximately a 100 cm length of the analytical tape (24) enables the analyte concentration to be monitored over a period of about 24 hours.

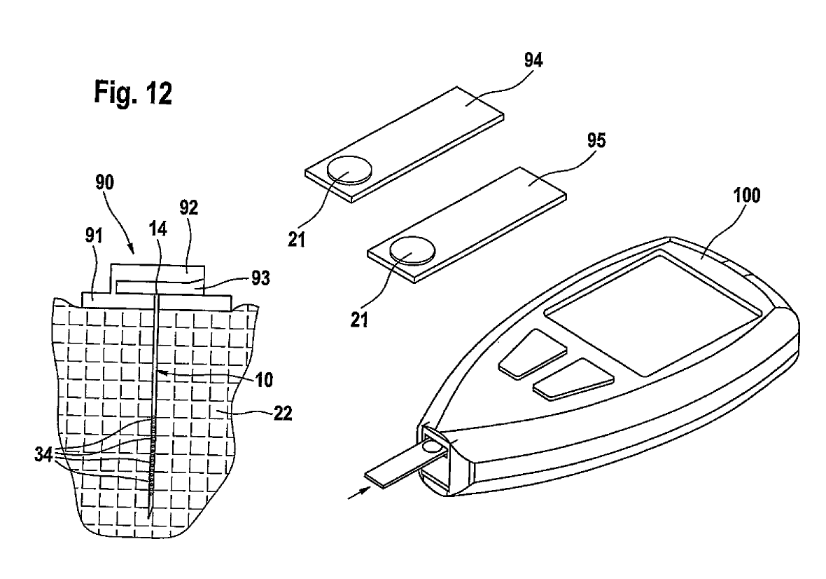

FIG. 12 shows a less integrated embodiment of a monitoring system using a catheter according to the invention. A unit (90) which can be worn on the body comprises a catheter (10) that can be implanted in body tissue (22) where the catheter (10) is held in a plate (91) which is attached to the body. A holder (92) for test elements (94) with a receiving opening (93) is located above the catheter opening (14). When a first test element (94) is inserted into the receiver (93) the analytical zone (21) is positioned above the catheter opening (14) and body fluid emerging from the catheter wets the analytical zone (21). When sufficient body fluid has been applied to the test zone which can for example be visually detected by the user, the test element is inserted manually into a conventional analyzer (100) and is evaluated there. If another measured value is required, the user can insert a second test element (95) into the opening (93) so that the analytical zone (21) is wetted. Although in such a system the user has to carry out more steps by himself than is the case for a system according to the previous figures, the embodiment of FIG. 12 has an extremely simple construction and it is possible to use conventional units that are freely commercially available for the test elements and evaluation instrument. The system according to FIG. 12 does not require the user to repeatedly puncture his body for the individual withdrawals of body fluid, rather the unit (90) provides the necessary body fluid for analysis according to needs.

While the invention has been taught with specific reference to the embodiments described above, one skilled in the art will recognize that changes can be made in form and detail without departing from the spirit and scope of the invention. The described embodiments are to be considered, therefore, in all respects only as illustrative and not restrictive. As such, the following claims, rather than the above description, define and illustrate the scope of the invention.

1-31. (canceled)

32. A catheter for monitoring the concentration of an analyte in a body fluid, the catheter including:
an outlet opening;
a catheter casing including at least one micro-opening sized to be permeable to at least part of the body fluid and to at least partially hold back corpuscular components of the body fluid; and
a catheter core at least partially located in the catheter casing.

33. The catheter according to claim 32 wherein the catheter core includes a surface and a microstructure formed on the surface, the microstructure defining a hollow space between the catheter core and catheter casing.

34. The catheter according to claim 33 wherein the microstructure includes micro-channels.

35. The catheter according to claim 33 wherein the microstructure includes a plurality of truncated cones.

36. The catheter according to claim 33 wherein the microstructure includes a plurality of truncated pyramids.

37. The catheter according to claim 33 wherein the microstructure includes a plurality of spherical calottes.

38. The catheter according to claim 33 wherein the catheter core includes a first microstructure and a second microstructure, the first microstructure located at an implantable distal area and the second microstructure located at a proximal area.

39. The catheter according to claim 38 wherein the first microstructure is shaped differently from the second microstructure.

40. The catheter according to claim 38 wherein the first microstructure covers substantially the implantable distal area in its entirety and the second microstructure covers a portion of the proximal area.

41. The catheter according to claim 32 wherein the catheter casing includes an inner wall and a microstructure formed on the inner wall.

42. The catheter according to claim 32 wherein a cross-section of the catheter casing is substantially round.

43. The catheter according to claim 32 wherein a cross-section of the catheter casing is substantially elliptical.

44. The catheter according to claim 32 wherein the catheter core lies coaxially within the catheter casing.

45. The catheter according to claim 32 wherein the catheter casing has a substantially rectangular profile and the catheter core has a complementary profile.

46. The catheter according to claim 33 wherein the microstructure includes axial grooves.

47. The catheter according to claim 33 wherein the microstructure of the catheter core includes transverse grooves.

48. The catheter according to claim 32 wherein the catheter core has a monolithic structure.

49. The catheter according to claim 34 wherein the micro-channels have a width between about 5 to about 20 μm.

50. The catheter according to claim 34 wherein the micro-channels have a depth between about 5 to about 20 μm.

51. The catheter according to claim 32 wherein the catheter casino includes a hollow area and the catheter core fills at least about 95% of the catheter casing.

52. The catheter according to claim 32 wherein the micro-openings have a diameter between about 0.01 μm to about 1 μm.

53. A system for monitoring the concentration of an analyte in a body fluid comprising:
a catheter having an implantable distal area and a proximal area including an outlet including:
a catheter casing with a plurality of micro-openings which are permeable to at least a part of the body fluid and at least partially impermeable to corpuscular components of the body fluid;
a catheter core at least in part located in the catheter casing; and
a detection area for detecting the analyte in the body fluid.

54. The system as set forth in claim 53 wherein the catheter core is located substantially coaxial with the catheter casing.

55. The system according to claim 53 wherein the peripheral surface of the catheter core has a microstructure defining a hollow space between the catheter core and catheter casing.

56. The system according to claim 55 wherein the microstructure includes micro-channels.

57. The system according to claim 53 wherein the catheter casing includes an inner wall and the inner wall includes a microstructure.

58. The system according to claim 53 further including a second detection area.

59. The system according to claim 58 wherein the detection areas are formed on a continuous test element.

60. The system according to claim 59 wherein the test element is a tape.

61. The system according to claim 53 further including means for generating a negative pressure in the catheter.

62. A method for producing a catheter for monitoring the concentration of an analyte in a body fluid including the steps of:
generating at least one micro-opening in a catheter casing, the at least one micro-opening being permeable to at least a part of the body fluid and at least partially impermeable to corpuscular components of the body fluid; and
assembling the catheter casing around a catheter core.

63. The method according to claim 62 further including the step of forming micro-channels in the catheter core by microform ablation with a laser.

64. The method according to claim 62 further including the step of forming micro-channels in the catheter core by electrochemically etching.

65. The method according to claim 62 further including the step of forming micro-channels in the catheter core by photolithographic material ablation.

66. A method for monitoring the concentration of an analyte in a body fluid including the steps of:
taking up body fluid in an implantable distal area of a catheter having a catheter casing with micro-openings permeable to at least a part of the body fluid and at least partially impermeable to corpuscular components;
transporting the body fluid from the distal area to a proximal area of the catheter;
contacting the body fluid with a detection area; and
detecting the analyte.

67. The method according to claim 66 further including the step of applying a negative pressure to transport the body fluid from the distal area to the proximal area of the catheter.

68. The method according to claim 66 wherein the body fluid in the catheter is transported at a delivery rate of about 10 nl/min and the body fluid is substantially conveyed in less than approximately 15 min from the distal area to the proximal area of the catheter.
